(19) United States
(12) Patent Application Publication
(10) Pub. No.: US 2007/0191926 A1
(43) Pub. Date: Aug. 16, 2007

(54) Stent pattern for high stent retention

(75) Inventors: Alexander Nikanorov, Palo Alto, CA (US); Keif Fitzgerald, San Jose, CA (US); Steven A. Tyler, Portola Valley, CA (US); Boris Anukhin, San Jose, CA (US)

Correspondence Address: FULWIDER PATTON LLP, HOWARD HUGHES CENTER, 6060 CENTER DRIVE, TENTH FLOOR, LOS ANGELES, CA, 90045, US

(73) Assignee: ADVANCED CARDIOVASCULAR SYSTEMS, INC.

(21) Appl. No.: 11/353,391

(22) Filed: Feb. 14, 2006

(51) Int. Cl. A61F 2/06

(52) U.S. Cl. 623/1.15

(57) ABSTRACT

An expandable stent has larger cells located at the distal end of the stent than in the body portion so that a catheter balloon can more easily protrude into the cells to increase stent retention relative to the balloon. The intravascular stent has a plurality of cylindrical rings connected by links, the spacing of which is a factor in defining the cell size. The stent can be compressed or crimped onto a balloon catheter to a very low profile and maintain a high degree of stent retention due to increased spacing between rings in the region of the distal end ring.

### BACKGROUND OF THE INVENTION

The invention relates to vascular repair devices, and in particular intravascular stents, which are adapted to be implanted into a patient's body lumen, such as an artery or coronary artery, or bile duct, to maintain the patency thereof. It is an important feature of the present invention to provide a stent structure that can be crimped onto a catheter to form a high degree of stent retention so that during delivery of the stent to a coronary artery or other vessel or duct the stent remains on the catheter.

Stents are generally tubular-shaped devices which function to hold open a segment of a blood vessel or other body lumen such as a renal or coronary artery. At present, there are numerous commercial stents being marketed throughout the world. While some of these stents are flexible and have the appropriate radial rigidity needed to hold open a vessel or artery, there typically is a tradeoff between flexibility and radial strength and the ability to tightly compress or crimp the stent onto a catheter so that it does not move relative to the catheter or dislodge prematurely prior to controlled implantation in a vessel.

What has been needed and heretofore unavailable is a stent pattern which has a high degree of flexibility so that it can be advanced through tortuous passageways and can be readily expanded, remain tightly crimped onto a balloon catheter during delivery, and yet have the mechanical strength to hold open the body lumen or artery into which it is implanted and provide adequate vessel wall coverage. The present invention satisfies this need. That is, the stent of the present invention has a pattern that increases stent retention on the catheter.

### SUMMARY OF THE INVENTION

The present invention is directed to a stent that has a pattern or configuration that permits the stent to be tightly compressed or crimped onto a catheter to provide an extremely high stent retention on the catheter. The stent of the present invention generally includes a plurality of cylindrical rings that are interconnected to form a plurality of cells. In one embodiment, there are less cells in the distal end rings than in the remaining rings, for example, there are two cells in the distal end rings and three cells in all other rings. The two cell pattern allows more balloon material to protrude into the cells during crimping thereby increasing stent retention relative to the catheter balloon.

In another embodiment, each of the cylindrical rings making up the stent have a proximal end and a distal end and a cylindrical plane defined by a cylindrical outer wall surface that extends circumferentially between the proximal end and the distal end of the cylindrical ring. Generally the cylindrical rings have a serpentine or undulating shape which includes at least one U-shaped element, and typically each ring has more than one U-shaped element. The cylindrical rings are interconnected by links which attach one cylindrical ring to an adjacent cylindrical ring. The links are highly flexible and allow the stent to be highly flexible along its longitudinal axis. In this embodiment, all of the connecting links are substantially straight and substantially parallel to the longitudinal axis of the stent. Since the links are substantially straight and the struts that connect the U-shaped elements or undulations are substantially straight, the stent can be compressed or crimped to a much tighter or smaller diameter onto the catheter which permits low profile delivery as well as a tight gripping force on the catheter to reduce the likelihood of movement between the stent and the catheter during delivery and prior to implanting the stent in a vessel or a bile duct. In order to further improve stent retention on the expandable member (or balloon), the gap between adjacent rings on the distal end of the stent is greater than the gap between the rings on the main body of the stent. Further, one or more distal end rings have two cells per ring while the main body of the stent has three cells per ring. Each of these structural features increases stent retention on the catheter balloon since the balloon can protrude into the gap and into the larger two cell structure to hold the stent onto the balloon.

In yet another embodiment, each of the cylindrical rings making up the stent have a proximal end and a distal end and a cylindrical plane defined by a cylindrical outer wall surface that extends circumferentially between the proximal end and the distal end of the cylindrical ring. Generally the cylindrical rings have a serpentine or undulating shape which includes at least one U-shaped element, and typically each ring has more than one U-shaped element. The cylindrical rings are interconnected by at least one connecting link which attaches one cylindrical ring to an adjacent cylindrical ring. The links are highly flexible and allow the stent to be highly flexible along its longitudinal axis. In order to further improve stent retention on the expandable member, the gap between adjacent rings on the distal end of the stent is greater than the gap between adjacent rings on the main body of the stent. Further, the two distal end rings are connected together with undulating links having a straight portion and a U-shaped bend (like a hinge). The undulating links may take various configurations but in general have at least one U-shaped bend. The undulating links can include bends connected by substantially straight portions wherein the substantially straight portions are substantially perpendicular to the stent longitudinal axis. The undulating links provide greater flexibility and more space between rings for better crimping onto the catheter expandable member. The U-shaped portion of the undulating links are perpendicular to the longitudinal axis of the stent thereby increasing stent retention relative to the balloon.

In a further embodiment, each of the cylindrical rings making up the stent have a proximal end and a distal end and a cylindrical plane defined by a cylindrical outer wall surface that extends circumferentially between the proximal end and the distal end of the cylindrical ring. Generally the cylindrical rings have a serpentine or undulating shape which includes at least one U-shaped element, and typically each ring has more than one U-shaped element. The cylindrical rings are interconnected by at least one connecting link which attaches one cylindrical ring to an adjacent cylindrical ring. The links are highly flexible and allow the stent to be highly flexible along its longitudinal axis. In this embodiment all of the connecting links are substantially straight and substantially parallel to the longitudinal axis of the stent. Since the links are substantially straight and the struts that connect the U-shaped elements or undulations are substantially straight, the stent can be compressed or crimped to a much tighter or smaller diameter onto the catheter which permits low profile delivery as well as a tight gripping force on the catheter to reduce the likelihood of movement between the stent and the catheter during delivery and prior to implanting the stent in the vessel or into a duct. In order to further improve stent retention on the expandable member (or balloon), the gap between adjacent rings on the distal end of the stent is greater than the gap between the rings on the main body of the stent. Further, one or more distal end rings have two cells per ring while the main body of the stent has three cells per ring. Each of these structural features increases stent retention on the catheter balloon. In this embodiment, the links connecting the distal end rings extend from a peak of one ring to a peak of an adjacent ring. By connecting the distal end rings peak to peak, the gap between the end rings is greater than the gap between adjacent rings on the body of the stent. Thus, the distal end ring structure increases stent retention on the catheter balloon since the balloon can more easily protrude into the gaps to hold the stent in place.

In one embodiment, each of the cylindrical rings making up the stent have a proximal end and a distal end and a cylindrical plane defined by a cylindrical outer wall surface that extends circumferentially between the proximal end and the distal end of the cylindrical ring. Generally the cylindrical rings have a serpentine or undulating shape which includes at least one U-shaped element, and typically each ring has more than one U-shaped element. The cylindrical rings are interconnected by at least one connecting link which attaches one cylindrical ring to an adjacent cylindrical ring. The links are highly flexible and allow the stent to be highly flexible along its longitudinal axis. The undulating portion of the link has an S-shape to further increase the gap between the distal end rings and the main body rings. The S-shaped link includes bends and straight portions, the straight portions being substantially perpendicular to the longitudinal axis of the stent. Both the increased gap between the distal end rings and the main body rings, and the straight portions of the S-shaped links being perpendicular to the longitudinal axis increase the stent retention on the balloon portion of the catheter. More specifically, the balloon can protrude into the increased gap area, and the straight portions that are perpendicular to the longitudinal axis of the stent resist longitudinal movement of the stent relative to the balloon. Further, the S-shaped portion of the undulating links act like a hinge to further increase longitudinal flexibility.

### BRIEF DESCRIPTION OF THE DRAWINGS

FIG. 1 is an elevational view, partially in section, of a prior art stent mounted on a rapid-exchange delivery catheter and positioned within an artery.

FIG. 2 is an elevational view, partially in section, similar to that shown in FIG. 1 wherein the prior art stent is expanded within the artery, so that the stent embeds within the arterial wall.

FIG. 3 is an elevational view, partially in section, showing the expanded prior art stent implanted within the artery after withdrawal of the rapid-exchange delivery catheter.

FIG. 4 is a plan view of a flattened stent of one embodiment of the invention which illustrates the pattern of the rings and links.

FIG. 5 is a partial plan view of the stent of FIG. 4 which has been expanded to approximately 3.0 mm inside diameter.

FIG. 6 is a plan view of a portion of the stent of FIG. 4 rolled into a cylindrical configuration and tightly crimped so that the various stent struts are either in close contact or contacting each other.

FIG. 7A is a plan view of a flattened stent of another embodiment of the invention which illustrates the pattern of the rings and links.

FIG. 7B is a partial plan view of the stent of FIG. 7A which has been expanded.

FIG. 7C is a portion of the stent of FIG. 7A that is illustrated in a cylindrical configuration and is tightly crimped or compressed.

FIG. 8A is a plan view of a flattened stent of another embodiment of the invention which illustrates the pattern of the rings and links.

FIG. 8B is a plan view of the flattened stent of FIG. 8A where the rings and links have been crimped or tightly compressed.

FIG. 8C is a plan view of a portion of the flattened stent of FIG. 8A illustrating the relationship of the U-shaped member to the undulating link prior to crimping the stent.

FIG. 9A is a plan view of a flattened stent depicting the pattern of the rings and links including S-shaped links.

FIG. 9B is a plan view of the flattened stent of FIG. 9A where the rings and links have been crimped or tightly compressed.

FIG. 9C is a portion of the flattened stent of FIG. 9A depicting the S-shaped undulating portion of the link when the stent is in a partially crimped or compressed configuration.

FIG. 10A is a plan view of a flattened stent depicting the pattern of the rings and links including S-shaped links.

FIG. 10B is a plan view of the flattened stent of FIG. 10A in a crimped or compressed configuration.

FIG. 10C is a partial plan view of the flattened stent of FIG. 10A depicting the S-shaped undulating portion of the link when the stent is partially crimped or compressed

FIG. 11 is an enlarged partial plan view depicting the variable radial thickness of a part of a cylindrical ring.

### DETAILED DESCRIPTION OF THE PREFERRED EMBODIMENTS

The present invention stent improves on existing stents by providing a stent pattern that greatly increases the retention force between the stent and the balloon on which it is mounted. The design of highly flexible interconnecting members and their placement relative to cylindrical rings provides for a tightly compressed stent onto a catheter thereby maintaining a high degree of stent retention on the balloon during delivery of the stent to a vessel or duct for implantation.

Turning to the drawings, FIG. 1 depicts a prior art stent 10 mounted on a conventional catheter assembly 12 which is used to deliver the stent and implant it in a body lumen, such as a coronary artery, peripheral artery, or other vessel or lumen within the body. The catheter assembly includes a catheter shaft 13 which has a proximal end 14 and a distal end 16. The catheter assembly is configured to advance through the patient's vascular system by advancing over a guide wire by any of the well known methods of an over the wire system (not shown) or a well known rapid exchange catheter system, such as the one shown in FIG. 1.

Catheter assembly 12 as depicted in FIG. 1 is of the well known rapid exchange type which includes an RX port 20 where the guide wire 18 will exit the catheter. The distal end of the guide wire 18 exits the catheter distal end 16 so that the catheter advances along the guide wire on a section of the catheter between the RX port 20 and the catheter distal end 16. As is known in the art, the guide wire lumen which receives the guide wire is sized for receiving various diameter guide wires to suit a particular application. The stent is mounted on the expandable member 22 (balloon) and is crimped tightly thereon so that the stent and expandable member present a low profile diameter for delivery through the arteries.

As shown in FIG. 1, a partial cross-section of an artery 24 is shown with a small amount of plaque that has been previously treated by an angioplasty or other repair procedure. Stent 10 is used to repair a diseased or damaged arterial wall which may include the plaque 26 as shown in FIG. 1, or a dissection, or a flap which are sometimes found in the coronary arteries, peripheral arteries and other vessels.

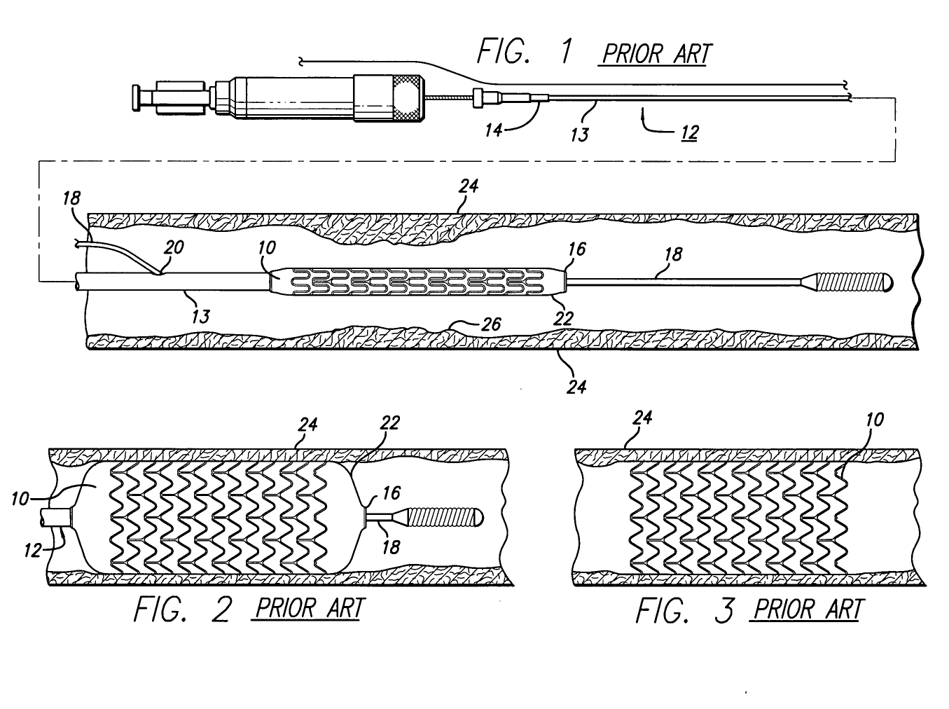

In a typical procedure to implant prior art stent 10, the guide wire 18 is advanced through the patient's vascular system by well known methods so that the distal end of the guide wire is advanced past the plaque or diseased area 26. Prior to implanting the stent, the cardiologist may wish to perform an angioplasty procedure or other procedure (i.e., atherectomy) in order to open the vessel and remodel the diseased area. Thereafter, the stent delivery catheter assembly 12 is advanced over the guide wire so that the stent is positioned in the target area. The expandable member or balloon 22 is inflated by well known means so that it expands radially outwardly and in turn expands the stent radially outwardly until the stent is apposed to the vessel wall. The expandable member is then deflated and the catheter withdrawn from the patient's vascular system. The guide wire typically is left in the lumen for post-dilatation procedures, if any, and subsequently is withdrawn from the patient's vascular system. As depicted in FIGS. 2 and 3, the balloon is fully inflated with the prior art stent expanded and pressed against the vessel wall, and in FIG. 3, the implanted stent remains in the vessel after the balloon has been deflated and the catheter assembly and guide wire have been withdrawn from the patient.

The prior art stent 10 serves to hold open the artery after the catheter is withdrawn, as illustrated by FIG. 3. Due to the formation of the stent from an elongated tubular member, the undulating components of the stent are relatively flat in transverse cross-section, so that when the stent is expanded, it is pressed into the wall of the artery and as a result does not interfere with the blood flow through the artery. The stent is pressed into the wall of the artery and will eventually be covered with endothelial cell growth which further minimizes blood flow interference. The undulating portion of the stent provides good tacking characteristics to prevent stent movement within the artery. While the present invention stent is sometimes described herein for use in a vessel, such as a coronary artery, the stent can be used in other body locations such as a bile duct.

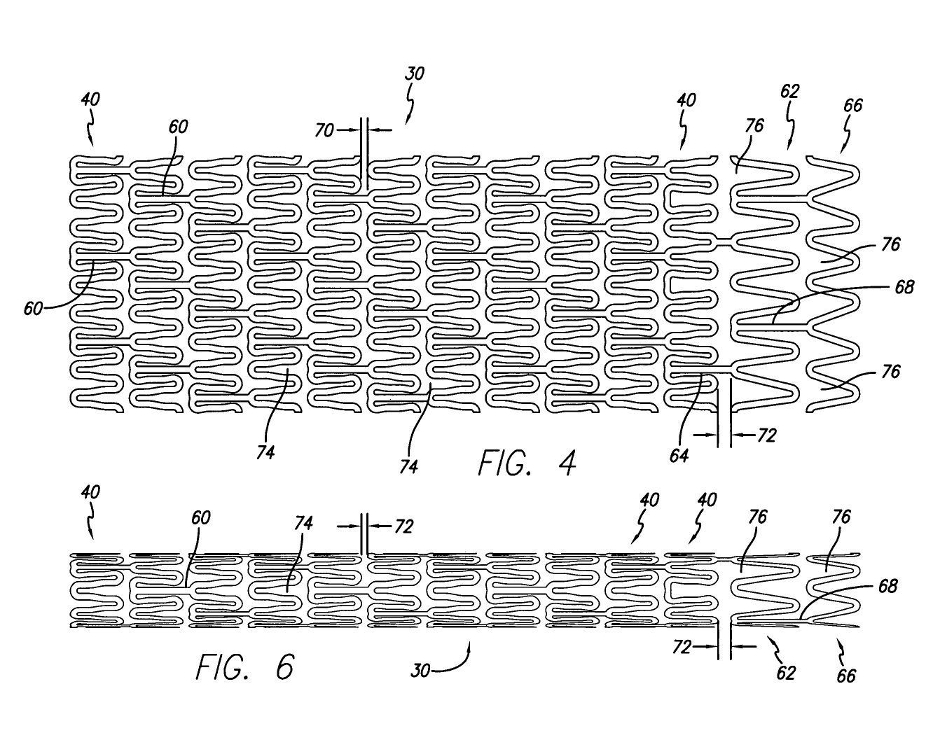

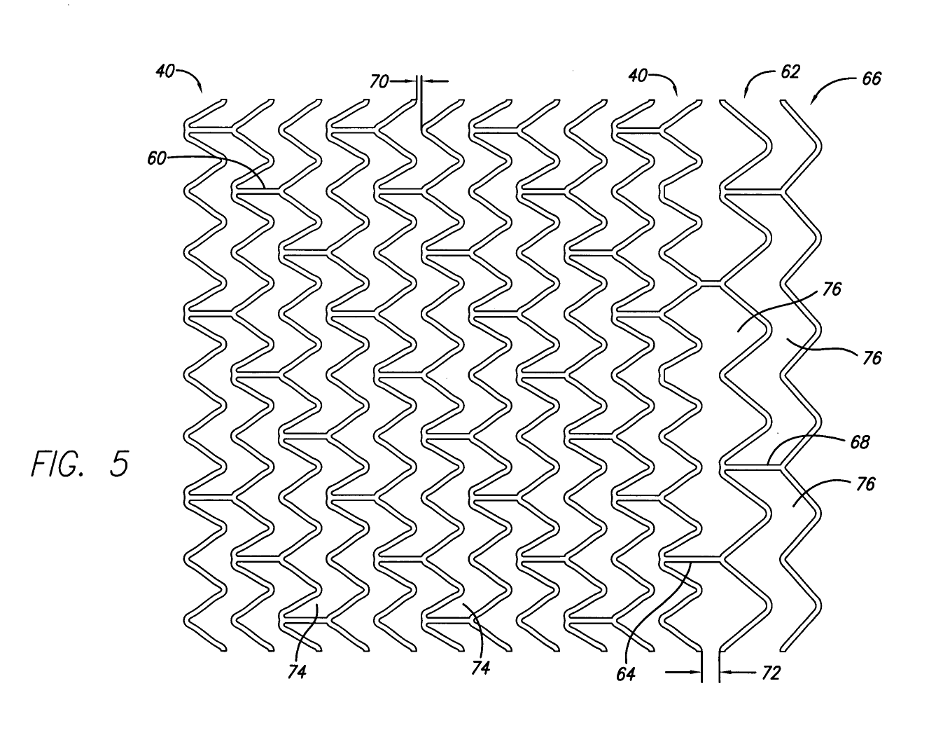

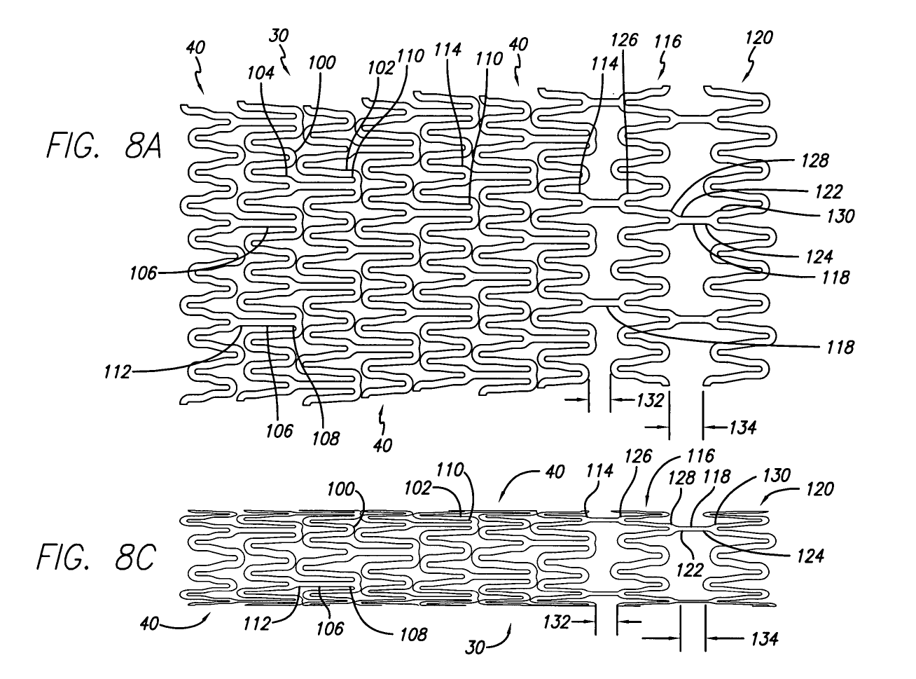

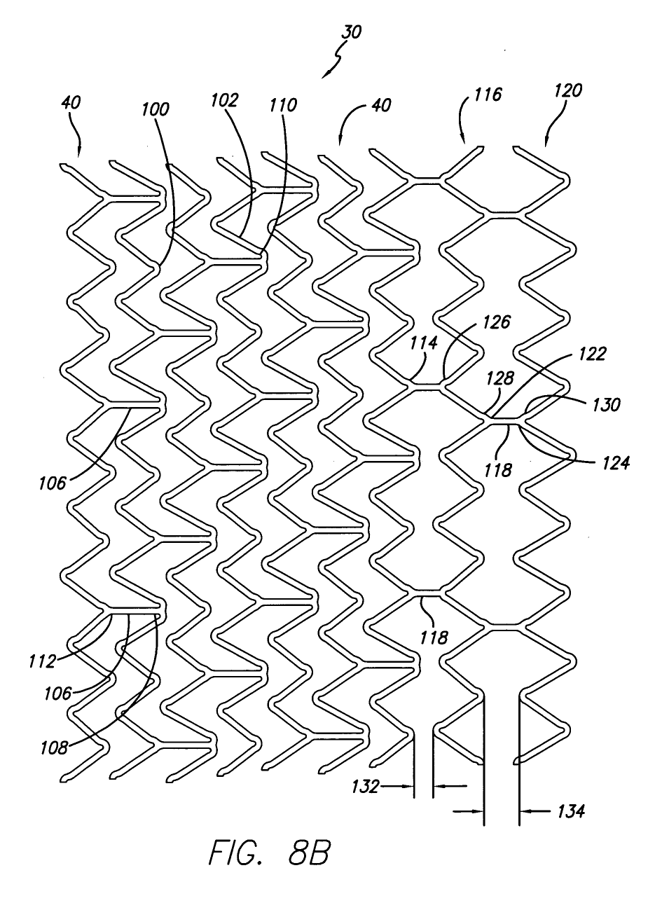

In keeping with the present invention, FIGS. 4-11 depict stent 30 in various configurations. The stent embodiments and patterns as disclosed herein are illustrative and by way of example only. The pattern can vary and still incorporate the stent retention features of the present invention. Referring to FIG. 4, for example, stent 30 is shown in a flattened condition so that the pattern can be clearly viewed, even though the stent is in a cylindrical form in use, such as shown in FIG. 6. The stent is typically formed from a tubular member, however, it can be formed from a flat sheet such as shown in FIG. 4 and rolled into a cylindrical configuration as shown in FIG. 6.

As shown in FIGS. 4-10C, stent 30 is made up of a plurality of cylindrical body rings 40 which extend circumferentially around the stent when it is in a tubular form (see FIGS. 6, 7C, 8C, 9C and 10C). Each cylindrical body ring 40 has a cylindrical ring proximal end and a cylindrical ring distal end. Typically, since the stent is laser cut from a tube there are no discreet parts such as the described cylindrical rings and links . However, it is beneficial for identification and reference to various parts to refer to the cylindrical rings and links and other parts of the stent as follows.

Each cylindrical body ring 40 defines a cylindrical plane which is a plane defined by the proximal and distal ends of the ring and the circumferential extent as the cylindrical ring travels around the cylinder. Each cylindrical ring includes a cylindrical outer wall surface which defines the outermost surface of the stent, and a cylindrical inner wall surface which defines the innermost surface of the stent. The cylindrical plane follows the cylindrical outer wall surface.

In keeping with the invention, FIGS. 4-6 show a stent 30 having cylindrical body rings 40 along a proximal portion of the catheter. The cylindrical body rings 40 are interconnected by first links 60 which are substantially straight and substantially aligned with the longitudinal axis of the stent. In this embodiment, there are three links spaced 120° apart connecting one cylindrical body ring to another cylindrical body ring. At the distal end of stent 30, a first distal end ring 62 is attached to an adjacent body ring 40 by one or more links that are substantially straight and substantially aligned with the longitudinal axis. In this embodiment, the first distal end ring 62 is attached to the adjacent cylindrical body ring 40 by two second links 64. A second distal end ring 66 is connected to the first distal end ring 62 by third links 68. In this embodiment, the second links and the third links are straight and substantially aligned with the longitudinal axis. Each of the second links and third links has a length that is substantially equal to each other but longer than first links 60 which connect adjacent cylindrical rings 40. The lengths of the second and third links may differ as long as they both are longer than the first lengths 60. Further, any of the first links 60, the second links 64, and the third links 68 can have a variable width in order to effect the flexibility of the stent along the longitudinal axis. If one of the links is substantially wider than another of the links, the flexibility will be less in the area around that link. In this embodiment, there are two third links 68 connecting the first distal end ring 62 to the second distal end ring 66.

The stent 30 shown in FIGS. 4-6 has improved stent retention when mounted on the expandable portion of a catheter for several reasons. First, in keeping with the invention, a first gap 70 is formed between the cylindrical body rings 40 along the stent longitudinal axis. For example, the distance between the ring proximal end 46 and the adjacent ring distal end 48 defines the longitudinal distance of the first gap 70. A second gap 72 is formed by the longitudinal distance between one of the cylindrical body rings 40 and the first distal end ring 62, and a second gap 72 also is created between the first distal end ring 62 and the second distal end ring 66. In this embodiment, the second gap 72 is relatively greater than the first gap 70. The increased distance formed by the second gap 72 allows a greater interaction between the distal end of the stent and the expandable portion (the balloon) of the catheter by allowing the protrusion of balloon material between the distal end rings to act as an anchor for the entire stent. In other words, there is more space between the distal end rings than the body rings in order to allow more balloon material to form or protrude into the space when the stent is crimped on to the balloon. Secondly, the cylindrical body rings 40 have three cells 74 between adjacent rings, while the first distal end ring 62 and the adjacent cylindrical body ring 40 have two cells 76 between the adjacent rings. Further, there are two cells 76 between the first distal end ring 62 and the second distal end ring 66. The number of cells is a direct function of the number of connecting links between adjacent rings. The area covered by cells 76 is greater than the area covered by cells 74. Since more balloon material can form within the two cells 76 (relatively higher area) at the distal end of the stent than into the three cells 74 (relatively lower area) between the cylindrical body rings 40, the stent has a higher retention on the balloon at the distal end of the stent due to the two-cell structure. Not only does the two-cell 76 pattern allow more balloon material to protrude into the cell area during crimping, but the two-cell structure also is more flexible than the three-cell structure. The three-cell structure 74 includes three connecting links between adjacent rings, while the two-cell structure 76 has only two connecting links between adjacent rings, thereby providing a more flexible distal region of the stent. In some embodiments, not show, there could be more than two distal end rings with a larger second gap 72.

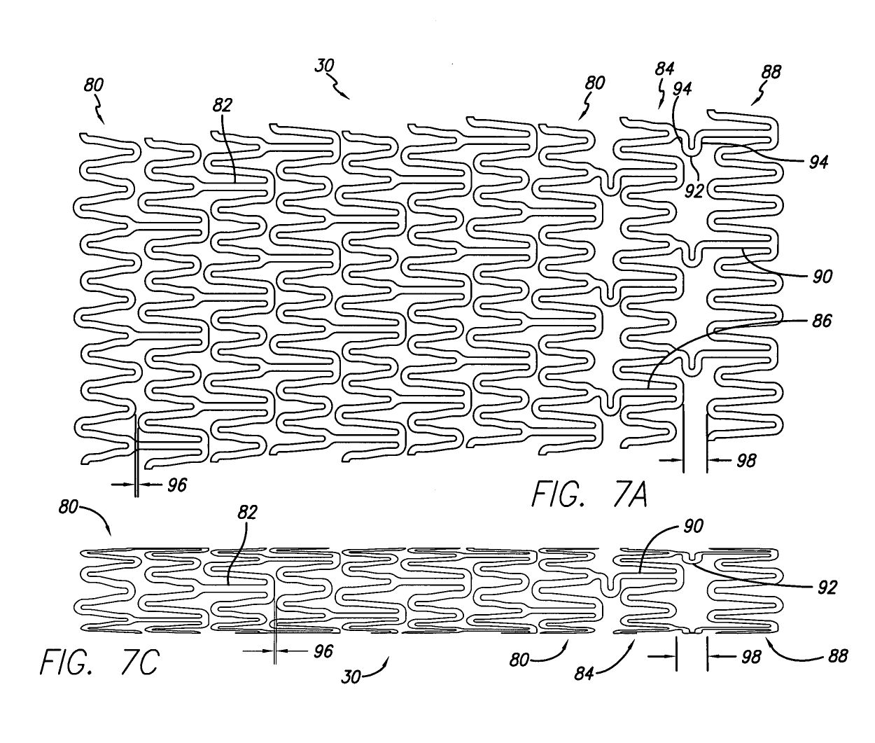

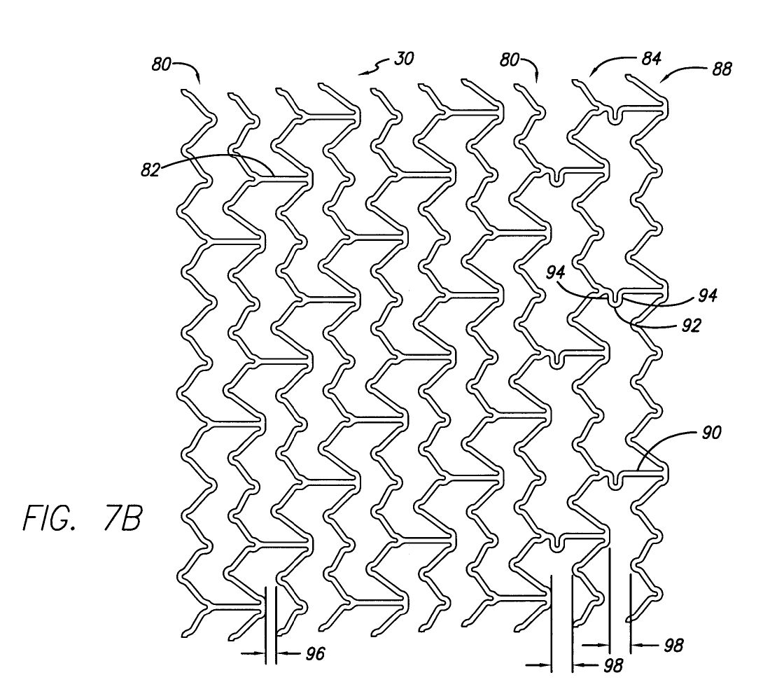

In another embodiment, as shown in FIGS. 7A-7C, stent 30 includes cylindrical body rings 80 interconnected by links 82 that are substantially straight and substantially aligned with the longitudinal axis. At the distal end of the stent, a first distal end ring 84 is attached to one of the cylindrical body rings 80 by first undulating links 86. Similarly, a second distal end ring 88 is attached to the first distal end ring 84 by second undulating links 90. Each of the undulating links connects cylindrical rings and contribute to the overall longitudinal flexibility of the stent due to their unique construction. The flexibility of the undulating links derives in part from curved portion 92 which acts as a hinge and is connected to straight portions 94 that are substantially straight and substantially perpendicular to the longitudinal axis of the stent. Thus, as the stent is being delivered through a tortuous vessel, such as a coronary artery, the curved portions 92 and the straight portions 94 of the undulating links 86,90, will permit the stent to flex in the longitudinal direction which substantially enhances delivery of the stent to the target site. With the straight portions being substantially perpendicular to the stent longitudinal axis, the undulating links 86,90 act much like a hinge at the curved portion to enhance flexibility. In this embodiment, there are three first undulating links 86 connecting the first distal end ring 84 to an adjacent cylindrical body ring 40, and three second undulating links 90 connecting the second distal end ring 88 to the first distal end ring 84.

In the embodiment shown in FIGS. 7A-7C, stent retention with respect to the stent being crimped onto the expandable portion of the catheter (the balloon) is greatly enhanced for several reasons. First, when the stent is crimped, the straight portions 94 that are attached to the curved portion 92 of the undulating link, are substantially perpendicular to the longitudinal axis of the stent. By being perpendicular to the longitudinal axis of the stent, that portion of the undulating link increases the dislodgment force required to pull the stent off of the balloon portion of the catheter. In addition, stent retention is increased by the larger gap between cylindrical rings in the distal end of the stent. In this embodiment, a first gap 96 is formed between the cylindrical rings 40 and a second gap 98 is formed between the first distal end ring 84 and an adjacent cylindrical body ring 40 as well as between first distal end ring 84 and the second distal end ring 88. The second gap 98 is greater than the first gap 96, thereby allowing more of the expandable member or balloon to project into the larger gap area when the stent is crimped onto the balloon. The more balloon that protrudes into the second gap area 98, the higher the retention force of the stent onto the balloon portion of the catheter.

In another embodiment, as shown in 8A-8C, the stent 30 can be described more particularly as having U-shaped portions 100, W-shaped portions 102, and Y-shaped portions 104. Although the stent is not divided into separate elements, for ease of discussion references to U-shaped portions 100, W-shaped portions 102, and Y-shaped portions 104 is appropriate. In this embodiment, the cylindrical body rings 40 are interconnected by links 106 that are substantially straight and substantially aligned with the longitudinal axis of the stent. The distal end 108 of the links is attached to valley 110 and form what appears to be W-shaped portion 102. The proximal end 112 of the links 106 is attached to first peaks 114 forming what appears to be the Y-shaped portion 104. The U-shaped portions 100 are unattached to any connecting link 106. In this embodiment, a first distal end ring 116 is attached to an adjacent cylindrical body ring 40 by links 118 that are substantially straight and substantially aligned with the longitudinal axis of the stent. Similarly, a second distal end ring 120 is attached to the first distal end ring by links 118. The proximal end 122 of the links 118 is attached to first peaks 114 and the distal end 124 of the links 118 are attached to second peaks 126 of the first distal end ring. Similarly, the proximal end 122 of links 118 are attached to third peaks 128 of the first distal end ring, and the distal end 124 of link 118 is attached to fourth peaks 130 of the second distal end ring 120. As can be seen, adjacent cylindrical body rings 40 are interconnected by links wherein the links are connected from a peak of one cylindrical ring to a valley of an adjacent cylindrical ring. In this manner, a first gap 132 is formed between adjacent cylindrical rings and is very small, on the order of less than 0.5 mm (0.0197 inch) and can range to as low as 0.1 mm (0.00394 inch). In contrast, the first distal end ring 116 is attached to the adjacent cylindrical body ring 40 by links 118 that are peak to peak, rather than peak to valley as with the body rings. Likewise, the first distal end rings 116 are connected by links 118 in a peak-to-peak pattern with the second distal end ring 120. A second gap 134 is formed between the first distal end ring 116 and the adjacent cylindrical body ring 40 as well as between the first distal end ring 116 and the second distal end ring 120. The second gap 134 is larger than the first gap 132 which, as previously described, provides a greater area for the expandable portion (balloon) of the catheter to protrude into when the stent is crimped onto the expandable portion of the catheter. This increases stent retention and prevents inadvertent stent dislodgment during delivery of the stent to, for example, the renal arteries or the coronary arteries.

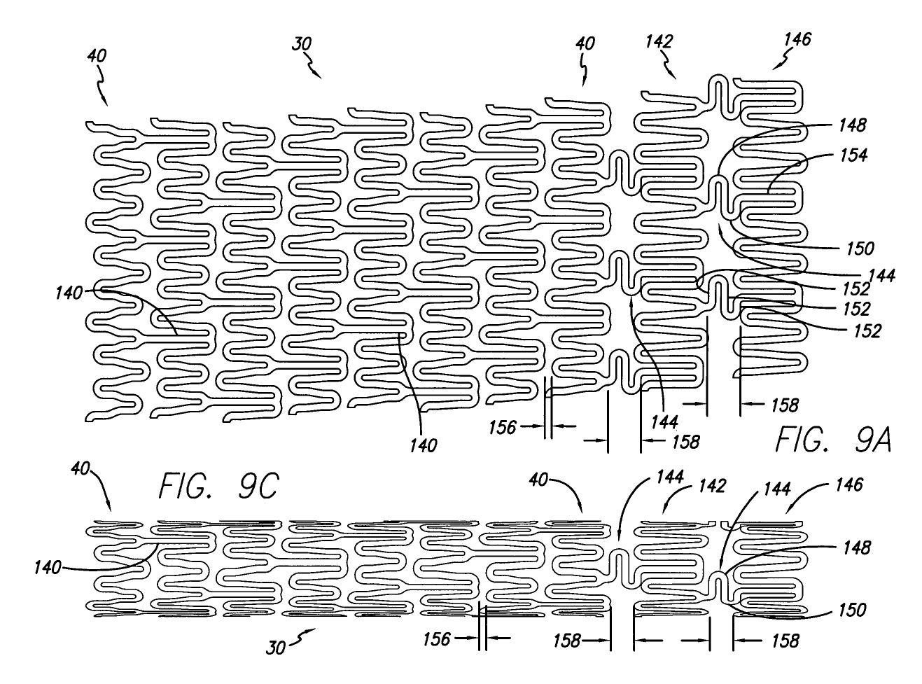

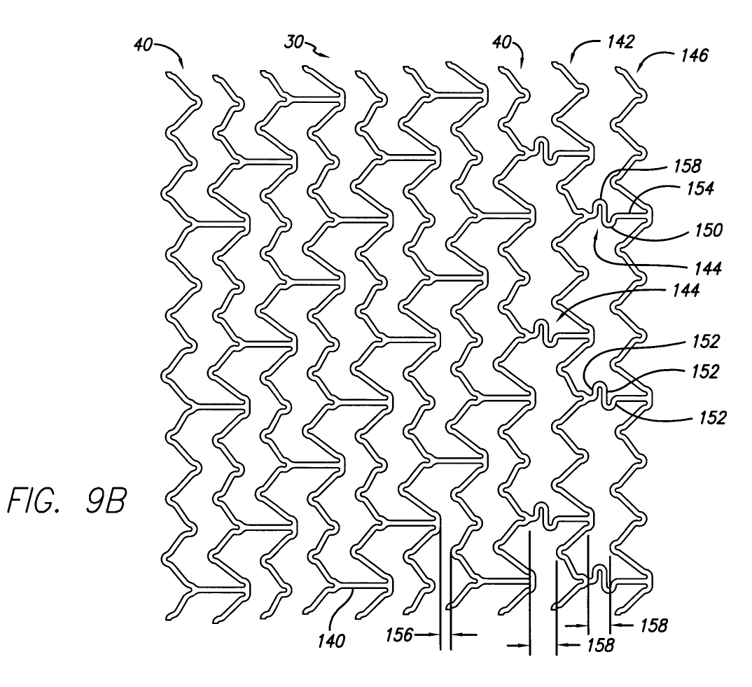

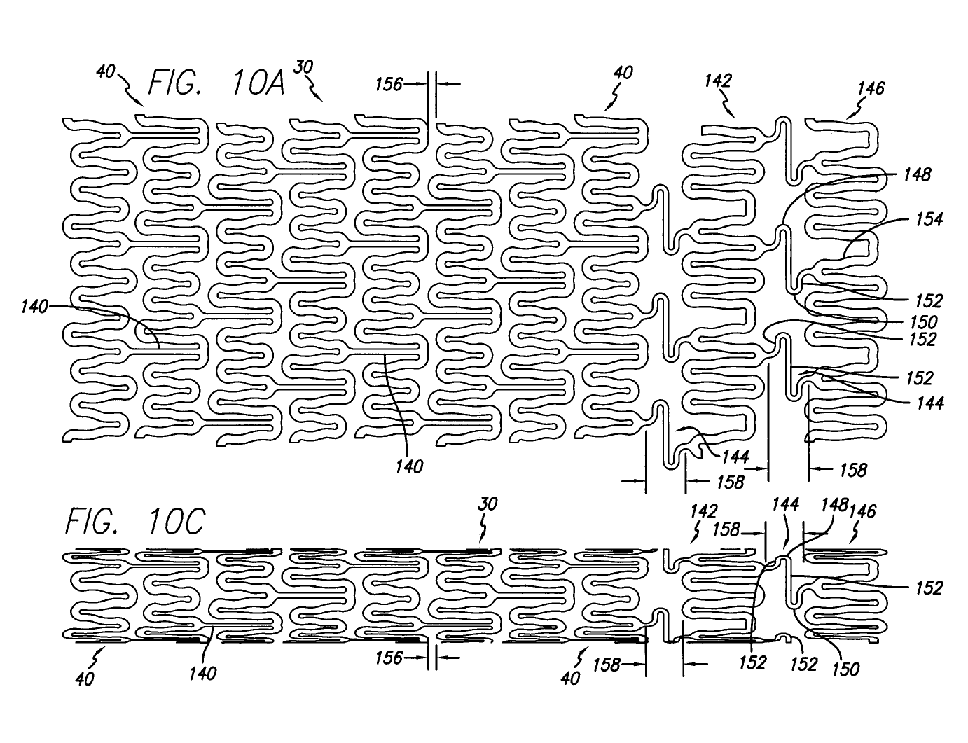

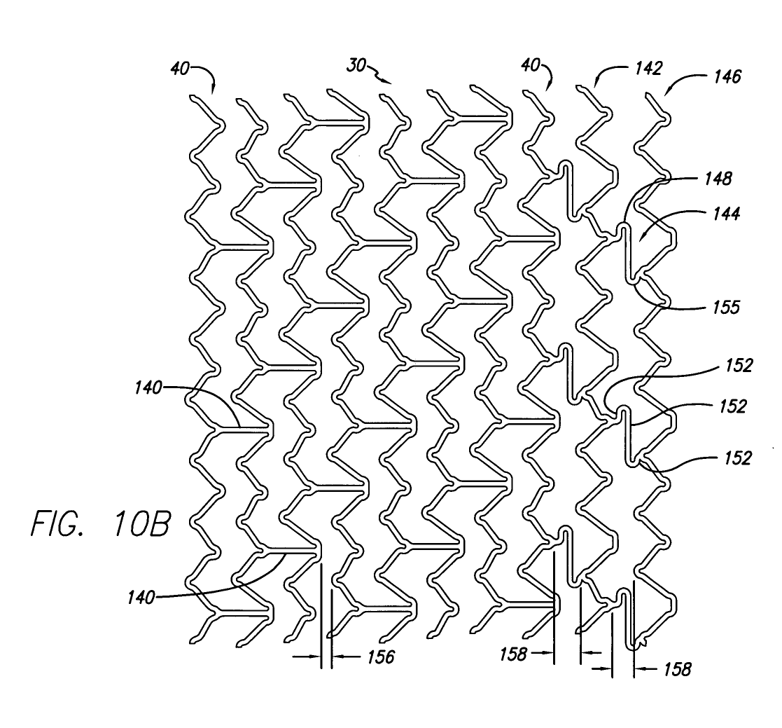

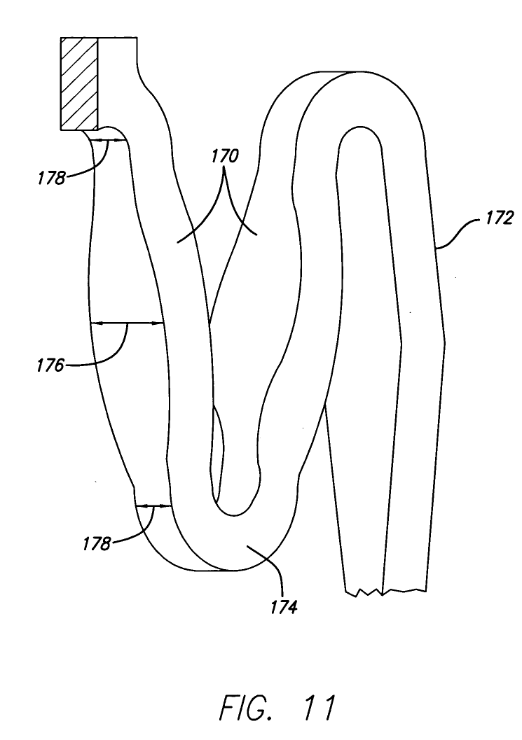

In another embodiment, shown in FIGS. 9A-10C, the stent 30 has a proximal portion having cylindrical body rings 40 that are interconnected by links 140 that are substantially straight and substantially aligned with the longitudinal axis of the stent. In this embodiment, a first distal end ring 142 is attached to an adjacent cylindrical body ring 40 by S-shaped links 144. Similarly, a second distal end ring 146 is attached to the first distal end ring by S-shaped links 144. In this embodiment, the S-shaped links 144 having a first bend portion 148 and a second bend portion 150 which are connected by substantially straight portions 152 that are substantially perpendicular to the longitudinal axis of the stent. A portion of the S-shaped links 144 have a connecting arm 154 that attaches to a portion of the first distal end ring 142 or a portion of the second distal end ring 146. The connecting arm 154 is substantially straight and substantially aligned with the longitudinal axis of the stent. The first bend portion 148 and the second bend portion 150 act as a hinge as the stent is being delivered through tortuous body lumens such as the renal arteries or the coronary arteries. In this embodiment, a first gap 156 is formed between the cylindrical body rings 40, and a second gap 158 is formed between the first distal end ring 142 and an adjacent cylindrical body ring 40, as well as between the first distal end ring 142 and the second distal end ring 146. The second gap 158 is larger than the first gap 156 which, as previously described, allows more of the balloon to protrude into the second gap 158 than the first gap 156 in order to provide greater retention of the stent on the balloon. Further, since the straight portions 152 of the S-shaped links 144 extend substantially perpendicular to the longitudinal axis of the stent, they have a tendency to resist dislodgment of the stent in the longitudinal direction and thereby provide greater retention force of the stent on the balloon. In the embodiment shown in FIGS. 10A-10C, the straight portions 152 of the S-shaped links 144 are somewhat longer than those disclosed in FIGS. 9A-9C. In the FIGS. 10A-10C embodiment, the second gap 158 is even larger than in the embodiments shown in FIGS. 9A-9C, which provides even more resistance to stent dislodgment, while providing enhanced flexibility. Further, since the straight portions 152 and FIGS. 10A-10C are somewhat longer, they provide a greater resistance yet to stent dislodgment since they are oriented perpendicular to the longitudinal axis of the stent. Again, as with the other disclosed embodiments, the number of distal end rings is not limited to two distal end rings, it could be more or less, as long as there is at least one larger gap like gap 158 near the distal end of the stent.

In another aspect of the invention, as shown in FIG. 11, the stent 30 is formed so that the various struts of the cylindrical rings, including the U-shaped portions, Y-shaped portions, W-shaped portions, and the links, all can be formed so that each has a variable radial thickness along the stent length. For example, the links may be radially thicker at one end than at the other end of the link. Further, first struts 170 and second struts 172 may vary in thickness (radial thickness) along their length in order to create variable flexibility in the rings. As shown in FIG. 11, peak 174 has first struts 170 that have radial thick portion 176 in the middle of the struts and radial thin portion 178 near the ends of the struts. As another example, the rings at, for example, the proximal end of the stent may be thicker radially than the rings in the center of the stent. A variable thickness stent that would benefit from the present invention is described and disclosed in U.S. Ser. No. 09/343,962 filed Jun. 30, 1999 and entitled VARIABLE THICKNESS STENT AND METHOD OF MANUFACTURE THEREOF (now abandoned), which is incorporated herein in its entirety by reference thereto. A variable thickness stent would benefit from the flexible nature of the present invention stent and still be crimped to a very low profile delivery diameter and still have high stent retention on the balloon as described herein.

The stent 30 of the present invention can be mounted on a balloon catheter similar to the catheter shown in the prior art device in FIG. 1. The stent is tightly compressed or crimped onto the balloon portion of the catheter and remains tightly crimped onto the balloon during delivery through the patient's vascular system. When the balloon is expanded, the stent expands radially outwardly into contact with the body lumen, for example, a renal or coronary artery. When the balloon portion of the catheter is deflated, the catheter system is withdrawn from the patient and the stent remains implanted in the artery. Similarly, if the stent of the present invention is made from a self-expanding metal alloy, such as nickel-titanium or the like, the stent may be compressed or crimped onto a catheter and a sheath (not shown) is placed over the stent to hold it in place until the stent is ready to be implanted in the patient. Such sheaths are well known in the art. Further, such a self-expanding stent may be compressed or crimped to a delivery diameter and placed within a catheter. Once the stent has been positioned within the artery, it is pushed out of the catheter or the catheter is withdrawn proximally and the stent held in place until it exits the catheter and self-expands into contact with the wall of the artery. Balloon catheters and catheters for delivering self-expanding stents are well known in the art.

The stent 30 of the present invention can be made in many ways. One method of making the stent is to cut a thin-walled tubular member, such as stainless steel tubing to remove portions of the tubing in the desired pattern for the stent, leaving relatively untouched the portions of the metallic tubing which are to form the stent. The stent also can be made from other metal alloys such as tantalum, nickel-titanium, cobalt-chromium, titanium, shape memory and superelastic alloys, and the nobel metals such as gold or platinum. In accordance with the invention, it is preferred to cut the tubing in the desired pattern by means of a machine-controlled laser as is well known in the art.

The stent of the present invention also can be made from metal alloys other than stainless steel, such as shape memory alloys. Shape memory alloys are well known and include, but are not limited to, nickel-titanium and nickel-titanium-vanadium. Any of the shape memory alloys can be formed into a tube and laser cut in order to form the pattern of the stent of the present invention. As is well known, the shape memory alloys of the stent of the present invention can include the type having superelastic or thermoelastic martensitic transformation, or display stress-induced martensite. These types of alloys are well known in the art and need not be further described here.

Importantly, a stent formed of shape memory alloys, whether the thermoelastic or the stress-induced martensite-type, can be delivered using a balloon catheter of the type described herein, or be delivered via a catheter without a balloon or a sheath catheter.

The present invention stent is ideally suited, for example, for drug delivery (i.e., delivery of a therapeutic agent) since it has a uniform surface area which ensures uniform distribution of drugs. Typically, a polymer is coated onto the stent of the type disclosed in U.S. Pat. Nos. 6,824,559 and 6,783,793 which are incorporated herein by reference.

These bioactive agents can be any agent, which is a therapeutic, prophylactic, or diagnostic agent. These agents can have anti-proliferative or anti-inflammmatory properties or can have other properties such as antineoplastic, antiplatelet, anti-coagulant, anti-fibrin, antithrombonic, antimitotic, antibiotic, antiallergic, antioxidant as well as cytostatic agents. Representative embodiments of the active component include actinomycin D (available from Sigma-Aldrich; or Cosmegen® available from Merck) or derivatives, analogs or synonyms thereof, such as dactinomycin, actinomycin IV, actinomycin I₁, actinomycin X₁, and actinomycin C₁; podophyllotoxins such as etoposide and teniposide (Bristol Myers Squibb and Sigma Chemical); cephalotin (Bristol Myers Squibb); trapidil; ticlopidine (Danbury Pharma, Genpharm); tranilast (SmithKline Beecham and LG Chemical Kissei, Japan); IIb-IIIa inhibitors such as eptifibatide (COR therapeutic); clobetasol (Glaxo Wellcome); COX-2 inhibitors such as celecoxib (CELEBREX) (Searle and Pfizer) and rofecoxib (VIOXX) (Merck); PGE1 or alprostadil (Bedford); bleomycin; ENDOSTATIN (EntreMed); ANGIOSTATIN (EntreMed); thalidomide; 2-methoxyestraidol (EntreMed and Sigma Chemical) curcimin (the major constituent of turmeric power extract from the rhizomes of the plant Curcuma longa L found in south and southeast tropical Asia); cisplatin (Sigma Chemical); dipyridamole; tirofiban; verapamil; vitronectine; argatroban; and carboplatin (Sigma Chemical). Additionally corticosteroids such as anti-inflammatory glucocorticoids including clobetasol, diflucortolone, flucinolone, halcinonide, and halobetasol can also be used. In one embodiment, faster acting non-steroidal anti-inflammatory agents such as naproxen, aspirin, ibuprofen, fenoprofin, indomethacin, and phenylbutazone can be used in conjunction with the glucocorticoids. The use of a non-steroidal anti-inflammatory agent is useful during the early stages of the inflammation in response to a mechanically mediated vascular injury. Examples of suitable therapeutic and prophylactic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules which bind to complementary DNA to inhibit transcription, and ribozymes. Some other examples of other bioactive agents include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy. Examples of anti-proliferative agents include rapamycin and its functional or structural derivatives, 40-O-(2-hydroxy)ethyl-rapamycin (everolimus), and its functional or structural derivatives, paclitaxel and its functional and structural derivatives. Examples of rapamycin derivatives include methyl rapamycin, ABT-578, 40-O-(3-hydroxy)propyl-rapamycin, 40-O-[2-(2-hydroxy)ethoxy]ethyl-rapamycin, and 40-O-tetrazole-rapamycin. Examples of paclitaxel derivatives include docetaxel. Examples of antineoplastics and/or antimitotics include methotrexate, azathioprine, vincristine, vinblastine, fluorouracil, doxorubicin hydrochloride (e.g. Adriamycin® from Pharmacia & Upjohn, Peapack N.J.), and mitomycin (e.g. Mutamycin® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, thrombin inhibitors such as Angiomax ä (Biogen, Inc., Cambridge, Mass.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacore® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl (4-amino-TEMPO), estradiol, anticancer agents, dietary supplements such as various vitamins, and a combination thereof. Examples of anti-inflammatory agents including steroidal and non-steroidal anti-inflammatory agents include tacrolimus, dexamethasone, clobetasol, combinations thereof. Examples of such cytostatic substance include angiopeptin, angiotensin converting enzyme inhibitors such as captopril (e.g. Capoten® and Capozide® from Bristol-Myers Squibb Co., Stamford, Conn.), cilazapril or lisinopril (e.g. Prinivil® and Prinzide® from Merck & Co., Inc., Whitehouse Station, N.J.). An example of an antiallergic agent is permirolast potassium. Other therapeutic substances or agents which may be appropriate include alpha-interferon, bioactive RGD, and genetically engineered epithelial cells. The foregoing substances can also be used in the form of prodrugs or co-drugs thereof. The bioactive agents also include metabolites of the foregoing substances and prodrugs of these metabolites. The foregoing substances are listed by way of example and are not meant to be limiting. Other active agents which are currently available or that may be developed in the future are equally applicable.

While the invention has been illustrated and described herein, in terms of its use as an intravascular stent, it will be apparent to those skilled in the art that the stent can be used in other body lumens. Further, particular sizes and dimensions, number of undulations or U-shaped portions per ring, materials used, shape of the connecting links, and the like have been described herein and are provided as examples only. Other modifications and improvements may be made without departing from the scope of the invention.

What is claimed:

1. A stent mounted on an expandable member of a catheter for use in a body lumen, comprising:
a plurality of cylindrical rings aligned along a common longitudinal axis and interconnected to form the stent, each cylindrical ring having a first delivery diameter and a second implanted diameter;
each cylindrical ring having a distal end and a proximal end and being spaced apart longitudinally to form a plurality of first cells; and
a distal end ring having a distal end and a proximal end and being spaced apart longitudinally from an adjacent ring to form a plurality of second cells for increasing stent retention relative to the expandable member.

2. The stent of claim 1, wherein the second cells are bigger than the first cells.

3. The stent of claim 1, wherein two distal end rings are spaced apart longitudinally to form a plurality of third cells.

4. The stent of claim 3, wherein the third cells are bigger than the first cells.

5. The stent of claim 3, wherein the third cells are substantially equal in size to the second cells.

6. The stent of claim 1, wherein there are fewer second cells between each distal end ring than first cells between adjacent rings.

7. The stent of claim 1, wherein there are two second cells between the distal end rings and an adjacent ring, and three first cells between adjacent rings.

9. The stent of claim 1, wherein the stent is formed from a tube.

10. The stent of claim 1, wherein the stent is formed from a metal alloy.

11. The stent of claim 11, wherein the stent is formed from any of the group of metal alloys consisting of stainless steel, tantalum, nickel-titanium, cobalt-chromium and titanium.

12. The stent of claim 1, wherein the stent is formed from a shape memory alloy.

13. The stent of claim 13, wherein the stent is formed from the group of shape memory alloys consisting of nickel-titanium and nickel-titanium-vanadium.

14. The stent of claim 1, wherein the stent is formed from a superelastic or pseudoelastic metal alloy.

15. The stent of claim 14, wherein the stent is formed from the group of superelastic or pseudoelastic metal alloys consisting of nickel-titanium and nickel-titanium-vanadium.

16. The stent of claim 1, wherein at least a portion of the stent has a variable thickness configuration.

17. The stent of claim 1, wherein the cylindrical rings are interconnected by substantially straight links being substantially aligned with the longitudinal axis.

18. The stent of claim 1, wherein the distal end rings are interconnected to the adjacent cylindrical ring by a substantially straight link being substantially aligned with the longitudinal axis.

19. The stent of claim 1, wherein the distal end rings are interconnected by undulating links comprising at least one curved portion connected to a substantially straight portion, the substantially straight portion being substantially perpendicular to the stent longitudinal axis.

20. The stent of claim 19, wherein the substantially straight portion of the undulating links is perpendicular to the stent longitudinal axis when the stent is in the first delivery diameter configuration.

21. The stent of claim 19, wherein the substantially straight portion of the undulating links is perpendicular to the stent longitudinal axis when the stent is in the second implanted diameter configuration.

22. The stent of claim 19, wherein of the undulating links further comprise a plurality of curved portions.

23. The stent of claim 19, wherein the undulating links are configured to provide flexibility to the stent.

24. The stent of claim 1, wherein at least a portion of the stent is coated with a therapeutic agent.

25. A stent mounted on an expandable member of a catheter for use in a body lumen, comprising:
a plurality of body rings aligned along a common longitudinal axis and interconnected to form the stent;
each body ring being spaced apart longitudinally to form a first gap; and
at least two distal end rings being spaced apart longitudinally to form a second gap for increasing stent retention relative to the expandable member.

26. The stent of claim 25, wherein the second gap is greater than the first gap.

27. The stent of claim 25, wherein a third distal end ring is spaced apart longitudinally from the second distal end ring to form a third gap.

28. The stent of claim 26, wherein the third gap is greater than the first gap.

29. The stent of claim 26, wherein the third gap is substantially equal to the second gap.

30. The stent of claim 25, wherein each body ring includes peaks that are in phase with peaks of adjacent body rings.

31. The stent of claim 25, wherein the rings are configured to provide flexibility to the stent.

32. The stent of claim 25, wherein the stent is formed from a tube.

33. The stent of claim 25, wherein the stent is formed from a metal alloy.

34. The stent of claim 33, wherein the stent is formed from any of the group of metal alloys consisting of stainless steel, tantalum, nickel-titanium, cobalt-chromium and titanium.

35. The stent of claim 25, wherein the body rings are interconnected by substantially straight links being substantially aligned with the longitudinal axis.

36. The stent of claim 25, wherein the distal end rings are interconnected to the adjacent body ring by a substantially straight link being substantially aligned with the longitudinal axis.

37. The stent of claim 25, wherein the distal end rings are interconnected by undulating links comprising at least one curved portion connected to a substantially straight portion, the substantially straight portion being substantially perpendicular to the stent longitudinal axis.

38. The stent of claim 37, wherein the substantially straight portion of the undulating links is perpendicular to the stent longitudinal axis when the stent is in the first delivery diameter configuration.

39. The stent of claim 37, wherein the substantially straight portion of the undulating links is perpendicular to the stent longitudinal axis when the stent is in the second implanted diameter configuration.

40. The stent of claim 37, wherein of the undulating links further comprise a plurality of curved portions.

41. The stent of claim 37, wherein the undulating links are configured to provide flexibility to the stent.

42. A flexible intravascular stent mounted on an expandable member of a catheter for use in a body lumen, comprising:
a plurality of body rings aligned along a common longitudinal axis and interconnected to form the stent, each body ring having a first delivery diameter and a second implanted diameter;
each body ring having a distal end and a proximal end and being spaced apart longitudinally to form a first gap;
a first distal end ring and a second distal end ring each having a distal end and a proximal end and being spaced apart longitudinally from an adjacent body ring to form second gap, the second gap being greater than the first gap; and
the first distal end ring being interconnected to the second distal end ring with a plurality of substantially straight links.

43. A flexible intravascular stent mounted on an expandable member of a catheter for use in a body lumen, comprising:
a plurality of body rings aligned along a common longitudinal axis and interconnected to form the stent, each body ring having a first delivery diameter and a second implanted diameter;
each body ring having a distal end and a proximal end and being spaced apart longitudinally to form a first gap;
a first distal end ring and a second distal end ring each having a distal end and a proximal end and being spaced apart longitudinally from an adjacent body ring to form second gap, the second gap being greater than the first gap; and
the first distal end ring being interconnected to the second distal end ring with a plurality of undulating links.
